(19) United States
(12) Patent Application Publication
(10) Pub. No.: US 2010/0062538 A1
(43) Pub. Date: Mar. 11, 2010

(54) IDENTIFYING AND COUNTING PROTEINS IN A SAMPLE

(75) Inventors: GONGXIN YU, BOISE, ID (US); ERIC BROWN, BOISE, ID (US); HENRY A. CHARLIER, BOISE, ID (US)

Correspondence Address: PEDERSEN & COMPANY, PLLC, P.O. BOX 2666, BOISE, ID, 83701, US

(73) Assignee: BOISE STATE UNIVERSITY, BOISE, ID (US)

(21) Appl. No.: 12/536,476

(22) Filed: Aug. 5, 2009

Related U.S. Application Data
(60) Provisional application No. 61/086,697, filed on Aug. 6, 2008.

(51) Int. Cl. G01N 33/00

(52) U.S. Cl. 436/86

(57) ABSTRACT

The proteins in a cell are preferably proteolytically cleaved and chemically attached to another peptide of unique and known sequence. In one embodiment of the invention, peptide-linker-peptide triplets are synthesized with linker molecules such as polyhistidine. In a more preferred embodiment of the invention, peptide-mass differentiated group (MDG) constructs are synthesized. The MDG's may be obtained from a library of oligo-N(K)-peptides synthesized on resin beads, wherein N is the length of the peptides (with a default value of 4) and K is the number of alternative amino acids (with a default value of 10) at each position. Coupling between given peptides and linkers or MDG's creates recombinants with different overall masses that migrate separately in chromatographic separations. The peptides-linker/MGD's recombinants may be purified and sequenced by MS/MS analysis. The resulting purified and sequenced peptides are then counted, and the ratios of the different peptides within and/or between samples obtained.

This application claims priority of Provisional Application Ser. No. 61/086,697, filed Aug. 6, 2008, the entire disclosure of which is incorporated herein by this reference.

### BACKGROUND OF THE INVENTION

1. Field of the Invention

This invention relates generally to the field of proteomics which is the study of the entire complement of proteins (known as the “proteome”) found in a living cell, tissue or organism. More specifically, this invention relates to a method to quantitate (identify and count) proteins in a cell without the need for isotopic labeling.

2. Related Art

One aspect of proteomics is to quantitatively asses the various proteins found in a cell at different times under a variety of conditions. Such information may be used to better understand, for example, disease states or to identify targets for drugs for use in treating disease. Current proteomic techniques, including ICAT and iTRAQ, and acrylamide, ¹⁸0 and metabolic labeling techniques, use isotopic labeling of proteins and mass spectrometry to quantitate proteins. There is a need, however, for a method to quantitate proteins in a cell without isotopic labeling. This invention addresses that need.

### SUMMARY OF THE INVENTION

In the method of the present invention, the proteins in a cell are preferably proteolytically cleaved and chemically attached to another peptide of unique and known sequence. In one embodiment of the invention, peptide-linker-peptide triplets are synthesized with linker molecules such as polyhistidine. In a more preferred embodiment of the invention, peptide-mass differentiated group (MDG) constructs are synthesized. The MDG's may be obtained from a library of oligo-N(K)-peptides synthesized on resin beads, wherein N is the length of the peptides (with a default value of 4) and K is the number of alternative amino acids (with a default value of 10) at each position. Coupling between given peptides and linkers or MDG's create recombinants with different overall masses that migrate separately in chromatographic separations. The peptides-linker/MGD's recombinants may be purified and sequenced by MS/MS analysis. The resin serves a dual purpose; first, it may be used to synthesize the library of MDG molecules, and, second, the resin may be used to help purify the Tag-MDG's from the peptide mixtures. The resulting purified and sequenced peptides are then counted, and the ratios of the different peptides within and/or between samples obtained.

As a result, a series of modified peptides containing the unique and known sequence portion are generated. These modified peptides may be purified by a variety of conventional techniques, including nickel affinity chromotography, centrifugation, or filtration. The modified and purified peptides may then be identified, associated with a given protein in the proteome, and counted using conventional, preferably high—throughput mass spectroscopic methods in conjunction with conventional computational methods. This way, the different proteins in the cells of interest may be identified and their populations determined.

### BRIEF DESCRIPTION OF THE DRAWINGS

FIG. 1 is a schematic Serial Analysis of Protein Expression (SAPE) protocol outline for one embodiment of the invention.

FIG. 2 is a schematic modified SAPE protocol using N- and C-terminal trypsinized peptides (T^{nc}-SAPE protocol).

FIG. 3 is another schematic modified SAPE protocol using a library of peptide tags (Tag-SAPE protocol).

FIG. 4 is a schematic Basic Principles of Solid-Phase Peptide Synthesis outline for a preferred embodiment of the invention.

FIG. 5 is a schematic Synthetic Cycle of MDG-resin library creation for the preferred embodiment of the invention.

FIG. 6 is a schematic SAPE protocol outline to illustrate a seven-step procedure for the preferred embodiment of the invention.

### DETAILED DESCRIPTION OF EMBODIMENTS OF THE INVENTION

Mass spectrometry is the analytical technology of choice for many aspects of biomedical research and an emerging vital tool in early diagnosis, prognosis, monitoring disease progression or response to treatments. In such instances it is important to identify the proteins that are expressed at different amounts in the disease state or in response to treatments. Such information can be used to better understand the mechanisms that cause the disease and thereby providing critical information that could be used to improve treatment for the given condition. In order to detect such changes in protein expression levels, it becomes important to identify the protein and determine how much of it exists in each sample. Mass spectrometry is very powerful in determining the overall composition of unknown proteins. In spite of its power in identifying proteins, determining how much of each protein is present in a given sample remains a challenge. Several mass spectrometric techniques do exist for protein quantification. The commonly used methods in quantitative mass spectrometry are isotope coded affinity tags (ICAT), Isobaric tags for relative and absolute quantification (iTRAQ), acrylamide labeling, ¹⁸O-labeling during proteolysis, and metabolic labeling to incorporate ¹⁵N into peptides. All these techniques require differential stable isotope labeling that creates labeled and unlabeled fauns of a peptide with a mass shift. Drawbacks in these technologies, however, have prevented full potential of their application. For example, the cost and time required for creating and maintaining proteome quantification systems associated with metabolic labeling strategies are often incommensurate with the small amounts of the information obtained with these techniques. While iTRAQ quantitation is a powerful tool for comparing changes in protein expression, we have found it to be laborious and difficult to use. There are several steps in iTRAQ sample preparation that are conducted in parallel including purification and fractionation of proteins, protein digestion and iTRAQ labeling and slight differences in how these steps are accomplished can lead to differences in the final quantification values. Additionally, the numerous sample-handling steps in the protocol also result in unavoidable loss of sample. Finally, iTRAQ ratios can be overestimated if labeled peptides of slightly different mass end up being fragmented together and both contribute to the label peak intensities. Furthermore, isotopic labeling of proteins and its associated procedures can be very expensive, and such a cost greatly limits the scope of the work that can be done. An object of the present invention is to provide an isotope-free, quantitative mass spectrometry technique in protein analysis with a highly improved accuracy.

The present invention is inspired by the successful development and application of Serial Analysis of Gene Expression (SAGE). SAGE is a sequencing based high-throughput technology with a great accuracy in measuring gene expression through mRNA activity. SAGE does this by creating mRNA ‘tags’ that identify the transcript from which it came, and linking the tags together in a long chain for sequencing and analyzing. The relative abundance between tags in the chain should correspond to abundance of the transcript for which they code. According to the present invention, a mass spectrometric technique which is based on SAGE is described, namely Serial Analysis of Protein Expression (SAPE). SAPE acts on the same principles as SAGE: the ‘tags’ will be generated, linked, sequenced and finally counted. SAPE, however, differs from SAGE in several fundamental ways. First, the “tags” that will be used are trypsinized peptide fragments obtained from proteins in cellular extracts. These “tags” will be used to measure the dynamics of protein expressions, which is a more direct measurement of the actual functional state of a cell. Another difference between SAPE and SAGE is the tag-linker design itself. In one embodiment of SAPE the tags are connected through a special linker molecule to create peptide-linker-peptide triplets. While tags in SAGE (DNA) are directly sequenced, those (peptides) in SAPE have to be sequenced through mass spectrum analysis. The linker molecules in SAPE will be, for example, peptides such as polyhistidine that will serve a dual-purpose. First, the linker has a sequence that can be easily recognized, and can be used as a separator between two peptides in the triplets so that identities of the peptides can be clearly determined; second, the linker has features so that it can be used to affinity purify the peptide-linker constructs from the overall peptide mixture.

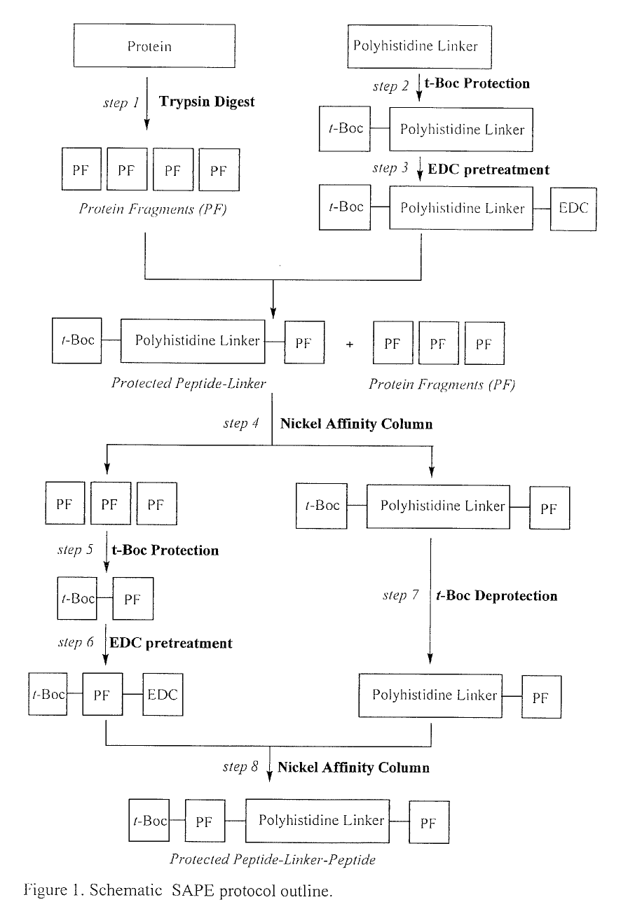

1) Construction of peptide-linker-peptide: FIG. 1 gives an overview of the experimental technique that will be used in one embodiment of the invention to form the peptide-linker-peptides. The SAPE technique for this embodiment will start with a trypsin digest of a protein mixture to generate the trypsinized peptide fragments (FIG. 1, step 1). Commercially available peptide fragments from at least two known proteins, such as C-reactive protein and bovine serum albumin (both available commercially), may be used. As needed, commercially available protein standards will be trypsinized and the resulting peptides will be used in the SAPE technique. Ultrafiltration of the peptide mixtures will be done to select peptides that are greater than 750 Daltons to ensure that peptides of at least 6 amino acids are present (It is the length of the shortest peptides identified from our MS/MS analysis). In parallel with processing the proteolytic peptides, tert-butoxycarbonyl (t-Boc) protection of the terminal amine of the polyhistidine linker peptide (available commercially) using di-tert-butyl dicarbonate will be performed (FIG. 1, step 2). Polyhistidine was selected as the linker because it will be easily purified using nickel affinity chromatography. It also is easily distinguished from any other peptide fragments in the peptide mixture and therefore the mass spectrum of the linker molecules will be clearly recognized. Protection of the terminal amine is necessary to prevent peptide-linker-peptides containing more than a single polyhistidine linker.

Next is coupling of the t-Boc-protected polyhistidine linker with the peptide fragments. The water-soluble carbodiimide, 1-ethyl-3-(3-dimethylaminopropyl)carbodiimide (EDC), may be used to activate the carboxyl group of the polyhistidine linker for attachment to the peptides in the proteolytic peptide mixture (FIG. 1, step 3). Since it is very important that coupling only occurs between the peptide fragment and the protected linker, pretreatment of the linker with stoichiometric amounts of EDC are required before addition of the peptide fragments. This precautionary step ensures no excess EDC remains and complete activation of the linker carboxyl group has occurred. The peptide-forming step requires substoichiometric addition of the EDC activated linker to the peptide fragments to ensure excess protein fragments remain for the second cycle. After the coupling reaction is completed, the polyhistidine-linker and excess peptide fragments may be purified using, for example, the nickel affinity column (FIG. 1, step 4). The resulting polyhistidine linked peptides may be dialyzed to remove imidazole introduced during the nickel chromatography step. Peptides unmodified by the polyhistidine linker will not bind the nickel column and these peptides may be collected and used in subsequent steps. These steps describe the completion of the first cycle, i.e., the attachment of a peptide fragment to the C-terminus of the linker.

The second cycle, is the random attachment of a peptide fragment to the N-terminus of the polyhistidine linker coupled peptide from cycle 1. First, the terminal amine of the protected peptide-linker may be deprotected with a strong acid (FIG. 1, step 7). Second, the unmodified peptide fragments that passed through the nickel affinity column without binding (described above) may be treated with Boc-protection and EDC activation steps in the same manner as in cycle one (FIG. 1, steps 5 and 6). Alternatively, 9-fluorenylmethyl carbamate (Fmoc) could be used as the amine-protecting group in place of t-Boc. The advantage of this approach is the Fmoc fluorescence may be used to easily identify the Fmoc-protected peptide-linker-peptide. The linker-peptide reacts with the EDC activated peptide-protected fragments. The Fmoc-protected (or t-BOC) peptide-linker-peptide may be purified, for example, by nickel affinity column chromatography before undergoing mass analysis with LC-ESI-MS/MS (FIG. 1, step 8).

2) Implementation of SAPE-specific database and computational algorithms: The MS/MS analysis may be performed according to, for example, the procedure described by Shibatani, T., David, L. L., McCormack, A. L., Frueh, K. and Skach, W. R. (2005) Proteomic analysis of mammalian oligosaccharyltransferase reveals multiple subcomplexes that contain Sec61, TRAP, and two potential new subunits. Biochemistry, 44. 5982-5992. The peptide-linker-peptide constructs may be filtered to remove particulates, and injected onto a 1 mm×8 mm trap column (Michrom BioResources, Inc) at 20 ml/minute in a mobile phase containing 0.1% formic acid. The trap cartridge may then be placed in-line with a 0.5 mm×250 mm column containing 5 mm Zorbax SB-C18 stationary phase (Agilent Technologies, Palo Alto, Calif.), and peptides are separated by a 2-30% acetonitrile gradient over 90 minutes at 10 ml/minute using a 1100 series capillary HPLC (Agilent). Peptides are analyzed using a LTQ linear ion trap fitted with an Ion Max Source and 34-gauge metal needle kit (ThermoFinnigan, San Jose, Calif.). Survey mass spectrometry (MS) scans may be alternated with 3 data-dependant MS/MS scans using the dynamic exclusion feature of the software to increase the number of unique peptides analyzed.

Data analysis: The next step is to search against protein sequence database using MS/MS spectra acquired from the analyses. The challenge is that the analytes in SAPE differ significantly from those in current MS/MS technologies. Unlike the spectrum of trypsinized peptides, those of the triplets cannot be used directly in SEQUEST-based database searches with ordinary protein databases. This problem is solved by two methods. First, SAFE-specific, SEQUEST searchable database of triplets (peptide-linker-peptides) may be created. The triplet in the database will cover all possible peptide pairs in the experimental population (in silico trypsinized peptides from all proteins in simple protein mixtures or whole proteomes of given organisms). Once the SAPE-specific database is made available, the MS/MS spectra may then be searched against it using the SEQUEST program (Thermo Finnigan, San Jose, Calif.). Identified peptide triplets are then filtered, collated and mapped to the triplet entries in the database using the program DTAselect. DTASelect will be configured to use Xcorr thresholds of 1.8, 2.5, and 3.5 for 1+, 2+ and 3+ parent ions, respectively, to select fully-tryptic peptide termini and to have a minimum DeltaCN value of 0.08. The peptides in the samples may be counted for protein quantification.

Building such SAPE-specific, SEQUEST searchable database is quite straightforward. For example, the database for a middle-sized bacterial genome of 3,000 proteins may be built with sequences of (3,000*20)² peptide-linker-peptides with an assumption of about 20 trypsinized peptides per protein. (3,000*20)² present all possible peptide pairs out of 3,000*20 peptides generated by trypsin-digestions. The databases will be exponentially increased with the size of the genomes and will become large when organisms of large genomes are analyzed. For example, we have to handle with a database of (40,000*20)² peptide-linker-peptide sequences (6.4×10¹¹) over 40,000 proteins in human proteome analysis. To meet the challenge, novel SAPE-specific algorithms are needed which will consider the predominantly observed patterns in the mass spectrum by the existence of polyhistidine within the triplets. Alternatively, de novo sequencing methods may be employed. These methods can infer a peptide sequence from spectrums without looking up a protein database, and, therefore, may be directly used in the SAPE analysis as soon as they can achieve a satisfactory performance.

Strategies for protein identification and quantification: One assumption in the SAPE development is that the peptide-linker-peptides are randomly generated: a process with an equal chance by which the constructs can be formed from trypsinized peptides that are either from the same proteins or from different ones. For example, if there are two proteins: X and Y in a protein mixture, protein X has 5 trypsinized peptides and protein Y has 3, there will be 25 different peptide pairs within protein X, 9 peptide pairs within protein Y and 30 peptide pairs between the two. The frequencies of particular peptides occurring in the triplets are, however, determined by the concentration of proteins from which the peptides came. A higher protein concentration would increase the possibility for peptides to form the constructs with their sister peptides (from the same proteins) as well as peptides from other proteins, and hence enhance their chance to be detected by MS/MS analysis. Peptide counts (the occurrences in the triplets) and the ratios calculated from these counts are, therefore, important indicators for relative expression levels. In case of protein X and Y, a simple formula as following can be used to calculate this ratio from the peptide counts:

(x₁+x₂+x₃+ . . . xₙ)/(y₁+y₂+y₃+ . . . +yₘ)*p_{y}/pₓ

Where x₁+x₂+x₃+ . . . xₙ and y₁+y₂+y₃+ . . . +yₘ are counts of individual peptides observed in MS/MS analysis for protein X and Y respectively; pₓ and p_{y} are the number of peptides from their in-silico trypsinization. Note: peptides that are shorter than 6 residues will preferably be eliminated from the in-silico trypsinizd peptide lists to be consistent with the experimental procedure described in the section of triplet synthesis.

Pitfalls and Innovative Schemes in SAPE protein quantification: SAPE acts on the same principles as SAGE and is designed to provide improved quantitative technology for proteomics research. The challenge is that the SAPE generates a peptide-linker-peptides mixture with a much-increased complexity. It is now (n*m)² compared to n*m prior to SAPE manipulation where n is number of gene in given genomes and m is the number of trypsinized peptides per proteins. The n varies from 3000 in bacterial genomes to 40,000 in human genome and m ranges from 1 to 120 or above depending on protein properties. The (n*m)² can hence create an immense complexity in a mixture of peptide-linker-peptides. In addition, SAPE leads to an inaccessibility of many synthesized triplets of peptide-linker-peptides by MS/MS technology. The useful limit for the type of MS/MS that we do called collision-induced dissociation is probably around 25 residues with 4000 Dalton (about 30 residues) as an up limit. With a linker of six residues, the peptide-linker-peptides would limit to peptides with a very narrow range in size, e.g. from 1 to 23 with an average of 12 residues. Lastly, the SAPE procedure requires a database with a similar complexity with additional sophistication in the afore-described algorithms in protein identification and quantification.

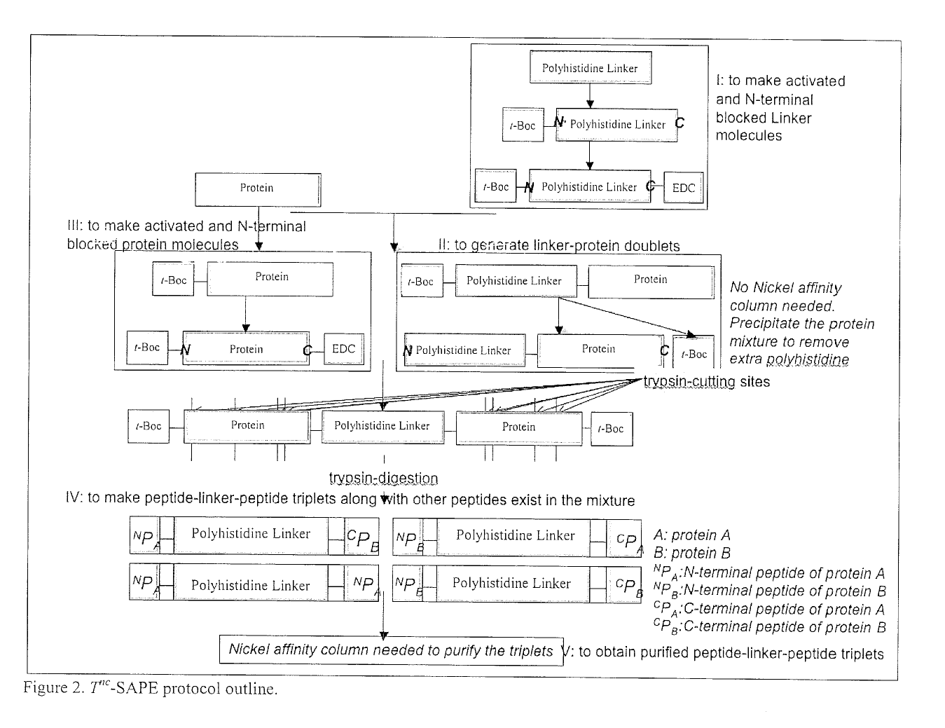

To address the complexities, we developed another SAPE protocol, named T^{nc}-SAPE, where T^{nc} represents N- and C-terminal trypsinized peptides (FIG. 2). T^{nc}-SAPE still follows SAPE procedure as described in FIG. 1 but with some modifications. Briefly, it starts with a procedure to make C-terminal-activated and N-terminal blocked Linker molecules (FIG. 2.I). The C-terminal-activated and N-terminal blocked Linker molecules are then coupled with undigested proteins to form protein-linker-protein molecules (FIGS. 2.II and 2.III). This is followed by enzyme digestions and nickel affinity column filtrations to get pure ^{N}P-linker-^{C}P where ^{N}P is the N-terminal peptides and ^{C}P is the C-terminal peptides derived from trypsin-digested proteins (FIG. 2.IV). The resulting peptide mixture has a complexity of (n*2)², a (m/2) fold decrease where m>=1 and m<=120.

One of the concerns is that how many proteins can be covered by the T^{nc}-SAPE technology. Through an in-house developed Pearl program, we found that in spite of the reduction in complexity, T^{nc}-SAPE still has higher protein coverage when compared to that of ICAT, one of the most popular MS technologies in protein quantification. By counting N- and C-terminal peptides that are small (<=15 amino acids that can be identified) and unique (one-to-one relationships between peptide and protein within the whole proteome), we found that T^{nc}-SAPE can detect 80.06% of the 3085 proteins at its maximum capability for the genomes of Brucella abortus biovar 1 str. 9-941, and the number decreases to 68.89% when ICAT is applied.

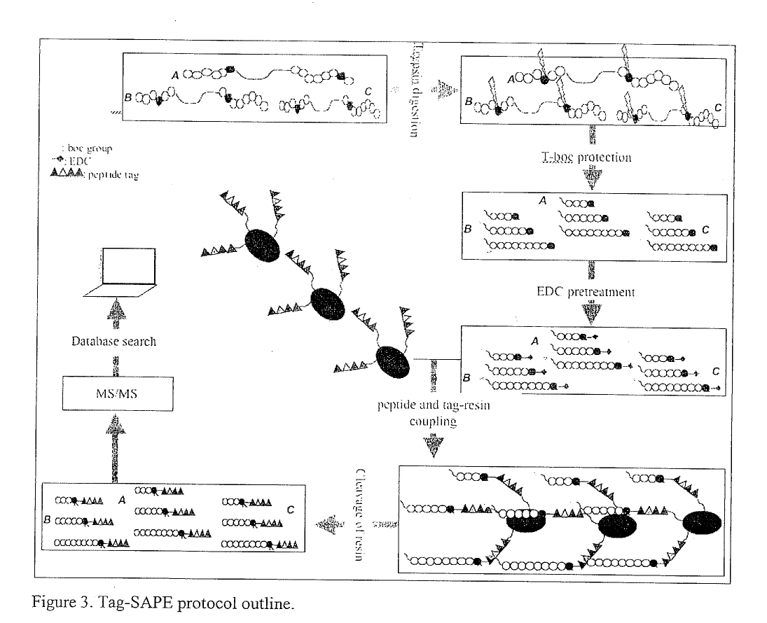

As in SAPE, T^{nc}-SAPE depends on the formation of peptide-linker-peptide. The size limitation for the peptides will still be an important consideration. Therefore, we developed a new scheme, called Tag-SAPE to further address the problem (FIG. 3). It follows the same strategy in SAPE but with a novel improvement. In this scheme, we first create a library of peptide tags. The tag is a resin-linked, N[M]-residue peptide (tetra-Resin) where N is length of the peptide tags and M is the number of amino acids at each position of the tags. The combination of N and M will thus generate a library of N^{M} tags in the library. The default numbers of the N and M are 4 and 10 but may vary depending on the protein complexity of experimental samples. The digested peptides will be then coupled to the tag-Resins to form peptide-tag-Resins. The peptide-tag-Resins are subsequently purified and peptide-tags are cleaved out from the resins for MS/MS analysis in this new Tag-SAPE scheme.

In both T^{nc}-SAPE and Tag-SAPE, special SEQUEST searchable database will be created for protein identification and quantification. To build these databases, all possible ^{N}P-linker-^{C}P in the case of T^{nc}-SAPE and peptide-tag-Resins in the case of tag-SAPE have to be created for whole proteome of target genomes. The peptides from MS/MS will then be identified and counted, finally the relative ratios of protein expressions calculated.

### PRELIMINARY RESULTS WITH THE PREFERRED EMBODIMENTS

Since its development in 1997, solid-phase peptide synthesis has been routinely used for the chemical synthesis of peptides and small proteins. In brief, an insoluble polymer support (resin) is used to anchor the peptide chain as each additional alpha-amino acid is attached. This polymer support, usually 20-50 μm diameter particles, is chemically inert to the reagents and solvents used in solid phase peptide synthesis. A labile group such as tBoc (tert-butyloxycarbonyl) and Fmoc (9-flourenylmethloxycarbonyl) protects the alpha-amino group of the amino acid. These groups can often be easily removed after each coupling reaction so that the next alpha-amino protected amino acid may be added. tBoc is stable at room temperature and easily removed with dilute solutions of trifluoroacetic acid (TFA) and dichloromethane. FMOC is a base labile protecting group that can be easily removed by concentrated solutions of amines (usually 20-55% piperidine in N-methylpyrrolidone). After synthesized, peptides are cleaved and purified.

One of the critical steps in SAPE is to create Tag-MDGs, the molecular construct of unique sizes, weights, biochemical properties, and retention time. Aqueous chemistry is essential for the objectives. However, the solid phase peptide synthesis described above is performed with organic solvents, which is known to be insoluble to many proteins or peptides. We predicted that we would confront such technological challenges in the development of the SAPE technology and have adapted a strategy to overcome this particular challenge. We first started with organic solvents for coupling reactions involving single amino acids and peptide mixtures of individual proteins. Then we moved into working with aqueous solvents.

Our initial resin used is trityl-chloride resin-His₁₀ (resin-His₁₀), which was customer-designed and synthesized by Peptides International, a Louisville-based peptide synthesis service company. The objective was to couple this resin with a single tBoc-protected glycine or a simple peptide mixture from trypsinzed ovalbumin in aqueous and/or organic solvents. The organic solvents we have tried so far include DCM, DMSO, acetonitrile and methanol. So far, the coupling efficiency was the best between resin-His₁₀ and tBoc-glycine in DCM. However, one potential drawback is that DCM is non-polar, which could cause solubility issues with peptides. Although some peptides could be quite hydrophobic, the digested ovalbumin is insoluble in DCM (data not shown). An experiment using a mixture of DCM and water was also not successful. In additional experiments with acetonitrile, we tested two different reaction temperatures: 50° C. and 70° C. Results from these experiments indicate that the 70° C. reaction worked better than the 50° C. reaction to a certain extent (close to 50% by peak height but without sufficient efficiency).

Coupling reactions between histidine and tBoc-glycine were successful in aqueous solvents, which indicates that we can achieve high-efficiency peptide synthesis in an aqueous environment. However, coupling reactions with resin-His₁₀ and tBoc-glycine were not successful, as none of the expected products were detected. Potential problems include the trityl-choloride resin and the trityl protection group in the side chain of histidine. The trityl-choloride resin is highly hydrophobic, which causes clotting of resin and the trityl protection group and significantly reduces the accessibility of His₁₀ in aqueous solvents. Two measures were taken to address this problem. First, we replaced the trityl-choloride resin-(His)₁₀ with H-(Gly)₄-CLEAR-Acid Resin (Cross-Linked Ethoxylate Acrylate Resin). According to the CLEAR product brochure, the entire cross-linked matrix of CLEAR is PEG-like (PEG: polyethylene glycol) in character and thus, hydrophilic CLEAR resins offer better swelling properties than the trityl-choloride resins in a wider variety of solvents (i.e., DCM, DMF, and water). This may lead to better coupling efficiencies and improved yields and purities. Importantly, CLEAR resins swell in aqueous systems, which provides a better starting material to develop the SAPE-specific procedure. Second, we used polyglycine instead of polyhistidine to avoid the complexity brought by the trityl protection group of polyhistidine. It is noteworthy to mention that the sole aim of using both trityl-choloride resin-His₁₀ and H-(Gly)₄-CLEAR-Acid Resin is to facilitate the development of SAPE. Ultimately, these resins will be replaced with a MDG library.

Subsequent experiments with the H-(Gly)₄-CLEAR-Acid Resin showed that no aggregation problems occurred in the aqueous solvent, and MS/MS analysis suggested promising results in coupling reactions. The coupling reactions between H-(Gly)₄-CLEAR-Acid Resin and tBoc-protected glycine generated (Gly)₅ in addition to some uncoupled (Gly)₄. However, puzzling results were obtained from coupling reactions between H-(Gly)₄-CLEAR-Acid Resin and trypsinized chicken ovalbumin peptides. Under the assumption that all peptide tags on the CLEAR-Acid Resin were (Gly)₄, and that their N-terminal amino groups were the only reactants to form peptide bonds with incoming peptides, all products should be peptide-(Gly)₄. Indeed, some sequences are the coupled products between (Gly)₄ and the trypsinized peptides (Table 1). Yet we also observed unexpected products such as (Gly)₂-peptides, (Gly)₃-peptides and even bare peptides. We hypothesize that the reactants at the H-(Gly)₄-CLEAR-Acid Resin are not homogeneous where H-(Gly)₃-CLEAR-Acid Resin and H-(Gly)₂-CLEAR-Acid Resin exist, and that other reactants also exist, most likely hydroxyl groups in unoccupied resin reaction sites.

TABLE 1
Products of coupling reactions between
H-(Gly)₄-CLEAR-Acid Resin and trypsinized
peptides (t-peptides) of chicken
ovalbumin protein.

| Sequence | | |
|---|---|---|
| ID # | Peptide Sequence | Peptide Mass |
| 98.15.42 | KLVNELTEFAKT | 1164.33 |
| 98.15.43 | KLVNELTEFAKT | 1164.33 |
| 98.15.52 | KLVNELTEFAKT | 1164.33 |
| 98.15.53 | KLVNELTEFAKT | 1164.33 |
| 98.15.56 | KLVNELTEFAKT | 1164.33 |
| 98.15.61 | KLVNELTEFAKT | 1164.33 |
| 98.15.89 | LVNELTEFAKGG | 1221.39 |
| 98.16.02 | LVNELTEFAKGG | 1221.39 |
| 11.15.42 | KLVNELTEFAKT | 1164.33 |
| 11.15.43 | KLVNELTEFAKT | 1164.33 |
| 11.15.52 | KLVNELTEFAKT | 1164.33 |
| 11.15.53 | KLVNELTEFAKT | 1164.33 |
| 11.15.56 | KLVNELTEFAKT | 1164.33 |
| 11.15.61 | KLVNELTEFAKT | 1164.33 |
| 100.16.37 | -SLHTLFGDELCK@ | 1437.62 |
| | GG | |
| 100.16.37 | K.SLHTLFGDELC* | 1437.59 |
| | K@V | |
| 100.16.38 | -SLHTLFGDELCK@ | 1437.62 |
| | GG | |
| 100.16.45 | K.SLHTLFGDELC* | 1437.59 |
| | K@V | |
| 100.16.45 | -SLHTLFGDELCK@ | 1437.62 |
| | GG | |
| 100.16.46 | -SLHTLFGDELCK@ | 1437.62 |
| | GG | |
| 100.16.65 | -SLHTLFGDELCK@ | 1437.68 |
| | GGG | |
| 147.14.24 | KHLVDEPQNLIKQ | 1306.49 |
| 147.14.34 | KHLVDEPQNLIKQ | 1306.49 |
| 147.14.37 | KHLVDEPQNLIKQ | 1306.49 |
| 147.14.45 | KHLVDEPQNLIKQ | 1306.49 |
| 147.14.56 | KHLVDEPQNLIKG | 1363.54 |
| | G | |
| 162.14.52 | F.SALTPDETY | 833.86 |
| 162.14.56 | P.CFSALTPDETY@VP | 1163.50 |
| 162.15.56 | L.TSPDETY@VP-KA | 1164.14 |
| 162.15.61 | LTSPDETYVP-K@A | 1164.14 |
| 162.19.90 | C.FS$ALTP-DETYVP- | 1713.74 |
| | K@GGG | |
| 162.20.04 | -PCFSALTPDETY@VPKGG | 1742.95 |
| 60.14.24 | K.HLVDEPQNLIKQ | 1306.49 |
| 60.14.34 | K.HLVDEPQNLIKQ | 1306.49 |
| 60.14.37 | K.HLVDEPQNLIKQ | 1306.49 |
| 60.14.45 | K.HLVDEPQNLIKQ | 1306.49 |
| 113.16.09 | F.Y*AP-ELLYY@ANKY | 1459.60 |
| 113.19.47 | F.YAPELLY@YANKY | 1362.53 |
| 113.19.99 | Y.YAPELLY*YANKGGGG | 1743.92 |
| 113.20.04 | Y.YAPELLY*YANKGGGG | 1743.92 |
| 113.20.04 | Y.YAPELLYY*ANKGGGG | 1743.92 |
| 140.22.18- | -DAFLGSFLYEY*SRGGGG | 1819.93 |
| 22.22 | | |
| 140.24.67 | -DAFLGSFLYEY*SRGGGG | 1819.93 |
| 14024.67 | -DAFLGSFLYEY*SRGGGG | 1819.93 |
| 140.24.86 | -DAFLGSFLYEY*SRGGGG | 1819.93 |
| 167.16.91 | K.QTALVELLKH | 1015.23 |
| 167.17.13 | K.QTALVELLKH | 1015.23 |
| 167.20.63 | E.LLKGGGG- | 601.72 |
| 75.14.52 | F.SALTPDET.Y | 833.86 |
| 75.14.56 | P.CFSALTPDETY@VP | 1363.50 |
| 75.15.56 | L.TSPDETY@VP-KA | 1164.14 |
| 75.15.61 | L.TSPDETYVP-K@A | 1164.14 |
| 104.20.35 | L.SHKGGGG- | 599.62 |
| 104.20.43 | L.SHKGGGG- | 599.62 |

One of the hallmarks of SAPE technology is the creation of peptide Tag-MDG constructs. The MDG is a peptide library with a pre-determined complexity so that coupling between given peptide tags and MDG will create constructs of unique sizes, weights, biochemical properties, and retention times, which can be subsequently sequenced by liquid chromatography electrospray ionisation tandem mass spectrometry (LC-ESI-MS/MS). Because the constructs are synthesized randomly between trypsinized peptides and MDGs, the frequencies of particular Tag-MDG constructs correspond to the expression levels of proteins from which the tags are derived. The relative levels of protein expression between two samples therefore can be inferred from peptide counts in these peptide-tag constructs.

### Synthesize a Library of Mass Differentiated Group (MDG) and Construct a Mixture of Tag-MDGs:

1. Synthesize MDG library: The objective is to create a library of peptides with a pre-determined complexity so that recombinants between the MDGs and peptide tags can provide the basis for peptide separation and quantification. The MDG is a resin-linked, N[M]-residue peptide (CLEAR-Resin), where N is length of the peptide tags, and M is the number of amino acids at each position of the tags. The combination of N and M will thus generate a library of N^{M} resin-peptide library. The default numbers of N and M are 4 and 10, respectively, but these values may vary depending on the protein complexity of proteome samples.

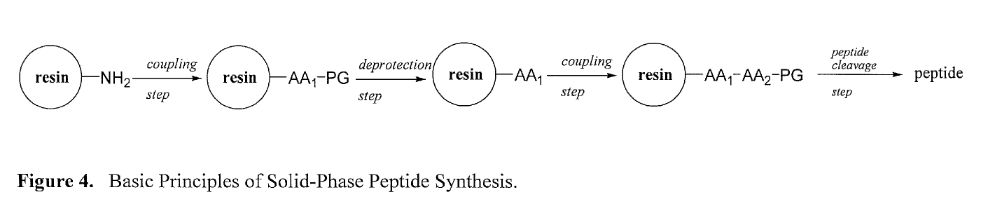

2. Solid-Phase Peptide Synthesis Technique Background: Solid-phase peptide synthesis (SSPS) techniques are routinely used for the chemical synthesis of peptides and small proteins and will be used to construct the peptide-tags used in this study. The general principles of solid-phase peptide synthesis are simple (FIG. 4). An insoluble polymer support (resin) is used to anchor the first amino acid residue via its carboxyl group to a hydroxyl or amino group on the resin. The growing peptide chain is then constructed using a sequence of repetitive steps. These steps involve deprotection of the polymer-bound amino acid N-terminus followed by activation/coupling of the next amino acid residue, whose N-terminus is protected to prevent coupling of free amino acid residues. The steps are then repeated until the desired peptide sequence is obtained. The final step is cleavage of the polypeptide from the insoluble polymer and its extraction into solution. See FIG. 4, entitled “Basic Principles of Solid-Phase Peptide Synthesis” wherein AA₁ is the first amino acid, AA₂ is the second amino acid and PG is protecting group.

Common protecting groups (PG) used in SSPS that block the α-amino group of the amino acid residue are tert-butyloxycarbonyl (tBoc) and 9-flourenylmethloxycarbonyl (Fmoc). These protecting groups are easily removed after each coupling reaction so that the next a-amino protected amino acid may be added to the polymer-bound polypeptide. Since the conditions used to remove the two protecting groups are different (Fmoc: high pH; tBoc: low pH), it is possible to protect side-chain functional groups and the N-terminus amino group with different protecting groups. Typically tBoc protecting groups are used to protect the side-chain functional groups that may infer with peptide elongation, while Fmoc is used to protect the N-terminus. An advantage of using Fmoc to protect the N-terminus is a dibenzofulvene-piperidine adduct that strongly absorbs in the UV range is formed upon Fmoc deprotection with piperdine, which allows quantification of the Fmoc deprotection step. Other reagents that are important in SSPS include the coupling reagents that chemically activate the carboxy moiety of the amino acid for peptide bond formation. Commonly used reagents are dicyclohexylcarbodiimide (DCC) and the water-soluble carbodiimide, 1-ethyl-3-(3-dimethylaminopropyl)carbodiimide (EDC).

Overall, there are numerous advantages to using the SSPS technique to construct the peptide-tags. First, physical loss of product is avoided since all the synthesis steps are performed in the same reaction vessel. Typical reaction vessels consist of a fritted syringe with a Luer lock, that allow the resin to be easily filtered and washed. Secondly, the coupling reactions can be driven to completion by using a large molar excess of protected amino acid. Finally, excess reagents and by-products are easily removed by thorough washings, eliminating difficult and time-consuming purification steps.

3. Choice of Supporting Resin: The choice of supporting resin is critical for the success of the proposed project. Numerous supporting resins are commercially available, which exhibit very different physical properties. The more traditionally used support resins for SPPS are the cross-linked polystyrene supports, which exhibit very good swelling properties in organic solvents (i.e. DCM and DMF). Swelling of the resin is an important property since 99% of the coupling sites on the resin bead are containing within the resin matrix and not at the surface. Unfortunately, as we discovered (see Preliminary Results Section), incomplete or no coupling resulted when couplings were performed in an aqueous solvent using a cross-linked polystyrene support. We believe this is the result of resin aggregation and incomplete swelling since the hydrophilic reaction conditions were incompatible with the hydrophobic polystyrene support. Therefore, we switched to the CLEAR resin, which does not possess a hydrophobic polystyrene core but instead contains a cross-linked ethoxylate acrylate resin that is much more hydrophilic and supports couplings in an aqueous environment. The ability to perform couplings in water is important for Specific Aim 1.2 since it involves creating Tag-MDG recombinants from trypsinized peptide fragments, which are only soluble in an aqueous environment. As a result, the CLEAR resin will be the support used to construct our resin-tag library.

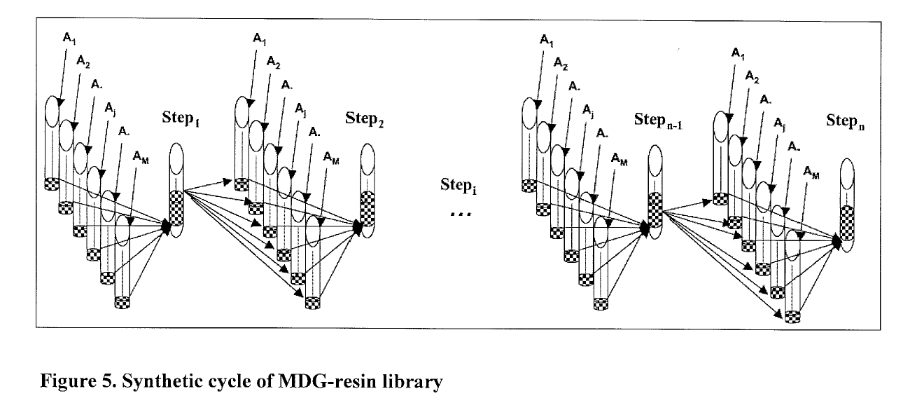

4. Resin-MDG Library Synthesis: Using solid-phase peptide synthesis techniques, a series of tetrameric peptides comprised of random amino acid sequences attached to a resin (resin-MDG library) will be synthesized. The synthetic procedure consists of a multi-step procedure involving multiple batches of coupling reactions followed by a merging of the coupled resin-products and then a dividing procedure (FIG. 5). For instance, in the first step of the procedure, which is attachment of the first amino acid residue (AA₁) to the resin, we want to make four batches of resin-AA₁, where AA₁ is a different amino acid for each batch. Therefore, if the procedure begins with 1.0 gram of resin then we will divide the resin into four batches, each with 0.25 gram of resin. Each of the four batches will be coupled to a different amino acid (AA₁) and then the four batches will be combined or merged together back into a single batch. This single batch, which now contains a mixture of resin-AA₁, where AA₁ could be four different amino acids, is then divided again into four batches. Each batch is deprotected (to remove the Fmoc group attached to the amino group of AA₁) and the second coupling step to add the second amino acid is performed. The merging and dividing steps are then repeated (FIG. 5, from step 2 to step n) until the randomized resin-bound tetrameric peptide (resin-MDG library) is constructed.

Each step involves a chemical coupling reaction to attach an amino acid to either the resin (step 1) or to the resin-peptide (from step 2 to step n). After each coupling step, resin-peptides from each different reaction or batch are merged, and then equally distributed to another set of reactions or batches. A deprotection step (to remove the Fmoc group from protected resin-peptide) is then performed. The steps are then repeated until the desired peptide sequence is obtained (from step 2 to step n). Aⱼᵢ in HAⱼᵢ-Resin are an Fmoc-protected residue where j is from 1 to m and i from 1 to n. m is the number of amino acids in each peptide position in the tag and n is the length of tag in the number of amino acids. The results are tag-Resin constructs: A_{1, j . . . m}-Resin from step 1, A_{2, j . . . m} A_{1, j . . . m}-Resin from step 2, A_{i, j . . . m} . . . A_{2, j . . . m} A_{1, i . . . m}-Resin from step i, A_{n-1, j . . . m} . . . A_{2, j . . . m} A_{1, j . . . m}-Resin from step n-1 and A_{n, j . . . m} . . . A_{2, j . . . m} A_{1, i . . . m}-Resin from the last step of the synthesis cycle. H in HAⱼᵢ-Resin represents a free N-terminal amino group.

5. Pitfalls/Alternatives in Resin-MDG Library Synthesis: The first concern is maximizing the loading of the first amino acid to the CLEAR resin. Often the addition of the first amino acid is the most difficult and lowest yielding step in SPPS. The simplest method to overcome this problem is to use a large excess of the activated amino acid, longer reaction times and repeat the coupling step with fresh reagents. Determination of the loading can then be determined by the Fmoc release method. The Fmoc release method allows the quantification of debenzofulvene-piperidine adduct formed after Fmoc deprotection by UV-vis spectroscopy. A comparison to the number of resin functionalities per gram of resin provided by the manufacturer will then allow the determination of the loading efficiency. If incomplete loading has occurred, any remaining coupling sites will be capped by an acetylation procedure using acetic anhydride.

The second concern is ensuring peptide synthesis is consistent at every step of the synthesis cycle and that complete coupling is occurring. Once again, the use of a large excess of reagents compared to the resin functionalities and longer reaction times are expected to drive the coupling reactions to completion. To test for incomplete coupling, a Kaiser test will be performed on a few resin beads prior to Fmoc deprotection. The Kaiser test (ninhydrin test) is a simple qualitative test that determines the presence of resin-bound free amines by observing whether the resin beads turn blue. A blue resin bead indicates free amines and incomplete coupling. If incomplete coupling occurs, then the coupling step will be repeated with fresh reagents until no free amino groups remain. In addition, the Fmoc release method could again be used to quantify the efficiency after each coupling step.

### The Synthesis of Tag-MDG Recombinants:

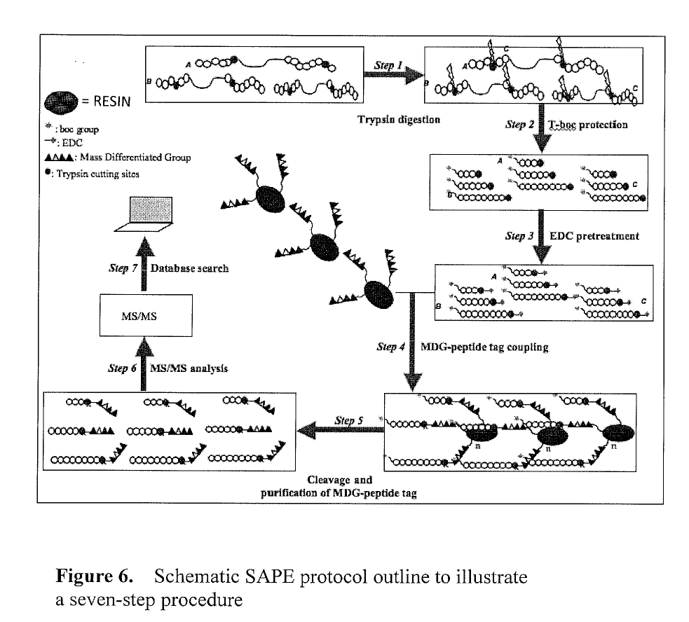

1. Synthetic Overview for Tag-MDG Recombinants: FIG. 6 gives an overview of the experimental technique that will be used to construct the Tag-MDG recombinants. The SAPE technique will start with a trypsin digestion of a protein mixture to generate the trypsinized peptide fragments (FIG. 6, step 1). In the initial stages of the project, commercially available proteins such as C-reactive protein and bovine serum albumin will be used until the protocol has been refined. As needed, the commercially available protein standards will be trypsinized and the resulting peptides will be used. After digested, the N-terminal of the trypsinized peptides will be protected by tert-butoxycarbonyl (tBoc) (FIG. 6, step 2). Protection of the terminal amine is necessary to prevent peptide-peptide reactions.

The next step is the coupling of the tBoc-protected peptides with the MDG-resin library. The water-soluble carbodiimide, 1-ethyl-3-(3-dimethylaminopropyl)carbodiimide (EDC), will be used to activate the carboxyl group of the tBoc-protected peptides (FIG. 6, step 3) (25). Since it is very important that coupling only occurs between the tBoc-protected peptides and the MDG-resin, pretreatment of the tBoc-protected peptides with stoichiometric amounts of EDC will be required before addition of the MDG-resin library. This precautionary step ensures no excess EDC remains and complete activation of the tBoc-protected peptides occurs (FIG. 6, step 4). After the coupling reaction is completed, the Tag-MDG-resin constructs will be treated with concentrated trifluoroacetic acid, which will simultaneously remove the tBoc protecting group and cleave the Tag-MDG recombinants from the resin (FIG. 6, step 5). The Tag-MDG recombinants will then be used in proteomic analysis with LC-ESI-MS/MS (FIG. 6, step 6) and compared to a SAPE-specific database (FIG. 6, step 7).

2. Pitfalls/Alternatives in Tag-MDG Synthesis: The primary concern when constructing the Tag-MDG recombinants is making sure the coupling steps go to completion in an aqueous solution. Prolonged reaction times are normally sufficient to overcome this problem. However, intramolecular and intermolecular aggregation of the resin-bound peptides via hydrogen bonding or hydrophobic interactions can prevent the accessibility of the reagents to the N-terminal amino group. Typically aggregation is only problematic when the resin-bound peptide contains more than five amino acid residues. As a result, the resin-MDG will be limited to five or less amino acid residues. Furthermore, the addition of detergent solvents, lithium chloride or solvents like dimethylsulfoxide (DMSO) or trifluoromethanol can be added to prevent aggregation.

SAPE acts on the same principles as SAGE and is designed to provide improved quantitative technology for proteomics research. The challenge is that SAPE generates a peptide Tag-MDG mixture with a much-increased complexity. It is now n*m*N compared to n*m prior to SAPE manipulation, where n is the number of proteins encoded in a given genome, in is the average number of trypsinized peptides per protein, and N is the number of MDGs in the MDG library. The n varies from 3000 in bacterial genomes to 40,000 in the human genome, whereas m ranges from 1 to 120 or above depending on protein properties. In addition, SAPE leads to an inaccessibility of some synthesized Tag-MDG by MS/MS technology. The useful limit for the type of MS/MS that we use (called collision-induced dissociation) is probably around 25 residues, with 4000 Dalton (about 30 residues) as an upper limit (private communication with Dr. Larry David, Oregon Health & Science University). With the addition of four-residue tags, the peptide tags would be limited to peptides with a very narrow range in size, e.g. from 7 to 26 residues.

An alternative approach is to develop a new SAPE protocol with reduced complexity. The approach is named T^{c}-SAPE, where T^{c} represents C-terminal trypsinized peptides. T^{c}-SAPE follows the general SAPE procedure described in FIG. 6 but with one modification. Briefly, it starts with a procedure to make C-terminal-activated and N-terminal protected proteins in a protein mixture. The C-terminal-activated proteins are then coupled with the MDG library to form MDG-protein constructs, followed by enzyme digestions and purification to obtain ^{C}P-MDG, where ^{C}P is the C-terminal peptides derived from trypsin-digested proteins. The resulting peptide mixture has a complexity of n*N, an m-fold decrease in the peptide complexity.

One concern is the uncertainty of how many proteins can be covered by the T^{c}-SAPE technology. Through an in-house developed Pearl program, we found that, in spite of the reduction in complexity, T^{c}-SAPE still has higher protein coverage than ICAT, one of the most popular MS technologies in protein quantification. By counting C-terminal peptides that are detectable (<=26 amino acids) and unique (one-to-one relationships between peptide and protein within the whole proteome), we found that T^{c}-SAPE can detect 2268 of the 3085 proteins (73.51%) for the genomes of Brucella abortus biovar 1 str. 9-941, whereas this number decreases to 2040 (66.13%) when ICAT is used. We achieved a similar phenomenon in the protein coverage in the genome of Bacillus anthracis str. ‘Ames Ancestor’ (3332 of 5309 proteins (62.76%) detectable by ICAT, as compared to 3537 (66.62%) using T^{c}-SAPE).

The MDG-resin library offers a great way to generate, purify, sequence and quantify proteins in complex proteomes. A critical step is to develop an environment where trypsinized peptides are coupled with MDG so that the number of MS/MS-sequenced peptides can accurately represent the protein expression profiles.

Although this invention has been described above with reference to particular means, materials and embodiments, it is to be understood that the invention is not limited to these disclosed particulars, but extends instead to all equivalents within the broad scope of the following claims.

We claim:

1. A method for determining populations of proteins, the method comprising:
obtaining proteins from a sample;
cleaving the proteins at known cut sites;
attaching unique and known peptides to the cleaved proteins at the cut sites from a random mixture of the peptides;
separating the resulting attached proteins-peptides from unattached proteins or peptides; and
analyzing the separated, attached proteins-peptides by mass spectronomy to identify and count them.

2. The method of claim 1 wherein the proteins are cleaved proteolytically.

3. The method of claim 1 wherein the separated, attached proteins-peptides are correlated to proteins in the sample.

4. The method of claim 1 wherein the unique and known peptides are attached to the cleaved proteins with a linker molecule.

5. The method of claim 4, wherein the linker molecule is polyhistidine.

6. The method of claim 1 wherein the unique and known peptides attached to the cleaved proteins comprise a mass differentiated group (MDG).

7. The method of claim 6, wherein the MDG comprises a resin.

8. The method of claim 7 wherein the resin is a bead.
